(19) United States
(12) Patent Application Publication
(10) Pub. No.: US 2023/0404777 A1
(43) Pub. Date: Dec. 21, 2023

(54) A MICROFLUIDIC SENSOR

(71) Applicant: CAMBRIDGE ENTERPRISE LIMITED, Cambridge (GB)

(72) Inventors: Sohini KAR-NARAYAN, Cambridge (GB); Qingshen JING, Cambridge (GB); Jehangir CAMA, Cambridge (GB); Liam IVES, Cambridge (GB); Vikas KHANDUJA, Cambridge (GB)

(73) Assignee: CAMBRIDGE ENTERPRISE LIMITED, Cambridge (GB)

(21) Appl. No.: 18/043,438

(22) PCT Filed: Aug. 27, 2021
(86) PCT No.: PCT/GB2021/052238

§ 371 Date: Feb. 28, 2023

(30) Foreign Application Priority Data
Aug. 28, 2020 (GB) 2013560.4

(51) Int. Cl. A61F 2/46; G01L 1/14; G01L 1/02; B81B 3/00; B81B 7/00; B81C 1/00

(52) U.S. Cl. CPC A61F 2/468; G01L 1/142; G01L 1/02; B81B 3/0075; B81B 7/0025; B81C 1/00674; B81C 2203/032; B81B 2203/0338; B81B 2203/04; B81B 2207/115; B81C 2201/0185; B81C 2201/034; B81B 2201/05

(57) ABSTRACT

A microfluidic sensor comprising: a first substrate; a second substrate; a cavity formed between the first substrate and the second substrate, the cavity comprising a reservoir portion and a channel portion extending from the reservoir portion; a capacitive element disposed between the first substrate and the second substrate, the capacitive element being at least partially disposed in the channel portion of the cavity; and a dielectric sensing liquid provided in the reservoir portion. Upon application of a force to the first substrate adjacent the reservoir portion, the reservoir portion is configured to deform and displace the sensing liquid along the channel portion, so as to change the capacitance of the capacitive element within the channel portion.

This invention relates to a microfluidic sensor, a method of manufacturing the same and a device comprising the same.

### BACKGROUND

Force sensing requirements are ubiquitous across a range of applications in the fields of biomedical engineering, robotic surgery, health monitoring and others. For example, during knee or hip replacement surgery, a surgeon will typically manually assess whether a joint implant is properly positioned by manually manipulating the limbs adjacent the joint and feeling for any unwanted resistance due to ligaments or bone structures over the expected range of motion. As this is a subjective assessment of whether a joint is balanced following fixation of the implant this is largely down to the skill of the individual surgeon. Should the joint feel imbalanced, the surgeon will reposition the implant, and reassess whether the joint is balanced. In total hip arthroplasty (THA) surgery, the correct balance of forces on the hip joint is essential for implant longevity and to prevent the need for revision surgery.

Existing force sensing devices can be based on resistive, capacitive, magnetic, optical, piezoresistive and piezoelectric detection modalities. However, designs involving magnetic or optical elements and their detection parts are bulky in volume and are limited by their ability to connect to electrical circuitry. Resistive and optical sensors involve significant power consumption. The choice of responsive piezoelectric materials is limited and thus their use is restricted when it comes to biocompatible applications.

Existing capacitive force sensors are typically used in low-load applications, such as tactile sensing and typically contain parallel-plate electrodes where the distance between the plates, and hence the measured capacitance, changes when an external load is applied. Such sensors often deform when load is applied which causes the electrodes themselves to deform. This is undesirable as the capacitance value is proportional to the effective overlap area of the electrodes, as well as the distance between them, and deforming the electrodes inherently changes the area between the electrodes as well as the distance between them. Furthermore, existing capacitive sensors typically exhibit a non-linear change in capacitance with applied force, resulting in variation in device sensitivity that introduces further difficulties when calibrating such sensors. While capacitive sensors are effective at low loads, their design makes them unsuitable for higher loads, such as those that are present in a human joint during normal daily activity or during joint replacement surgery.

Existing micro-fabrication processes for the development of microfluidic devices are commonly based on lithography, which is not cost-effective for scalability and are slow when prototyping complex geometries.

The present invention seeks to address at least some of these issues.

### BRIEF SUMMARY OF THE DISCLOSURE

Viewed from a first aspect, the present invention provides a microfluidic sensor comprising: a first substrate; a second substrate; a cavity formed between the first substrate and the second substrate, the cavity comprising a reservoir portion and a channel portion extending from the reservoir portion; a capacitive element disposed between the first substrate and the second substrate, the capacitive element being at least partially disposed in the channel portion of the cavity; and a dielectric sensing liquid provided in the reservoir portion. Upon application of a force to the first substrate adjacent the reservoir portion, the reservoir portion is configured to deform and displace the sensing liquid along the channel portion, so as to change the capacitance of the capacitive element.

Thus, the present invention provides a deformable microfluidic sensor that can be made from low-cost manufacturing processes and can be tuned to measure a desired range of forces.

The sensing liquid comprises a liquid that may have a relative permittivity of between 10 and 100.

The sensor may comprise an insulative coating disposed on a portion of the capacitive element. This advantageously protects the electrodes from corrosion due to direct contact with the dielectric liquid. A further advantage is the mechanical wear on the capacitive element is also reduced, which reduces the risk of the sensing element separating from the sensor, particularly when the sensor is bent and/or flexed in use. This insulative coating also provides a more robust sensor.

The reservoir portion may have a cross-sectional area between approximately 10 and 100 times greater than a cross-sectional area of the channel portion.

The capacitive element may be formed on a single surface of the channel portion. The capacitive element may comprise a first electrode extending from a first end to a second end and having a plurality of branches extending therefrom between the first end and the second end, and a second electrode extending from a first end to a second end and having a plurality of branches extending therefrom between the first end and the second end. The plurality of branches of the first electrode may be arranged to inter-digitate with the plurality of branches of the second electrode within the channel portion. This advantageously increases the sensitivity of the microfluidic sensor compared to a sensor with parallel electrodes having no branches.

The sensor may comprise at least one resiliently deformable member extending between the first and second substrates in the reservoir portion. The at least one resiliently deformable member may be formed as a well extending from the first substrate to the second substrate. This advantageously reduces the risk of the reservoir portion collapsing under loading.

The channel portion may extend from the reservoir portion to a distal end. The sensor may comprise a fluid port at the distal end.

Viewed from a further aspect, there is provided a device comprising: a first sensor as described above and configured to detect a first force applied at a first position on the device, and a second sensor as described above and configured to detect a second force applied at a second position on the device. The first sensor and second sensor may be configured to detect load in the same direction. Such a device advantageously provides a way to measure a distribution of forces across the device.

The first sensor may be configured to detect load in a first direction, and wherein the second sensor is configured to detect load in a second direction different to the first direction. This advantageously provides a way of determining the net direction and magnitude of the force being applied to the device.

The device may comprise: a first part, and a second part configured to receive at least a portion of the first part, such that when the portion of the first part is received within the second part, a gap is defined between the first part and the second part. In use, the reservoir portion of the first sensor and the reservoir portion of the second sensor may be disposed in the gap and are arranged to contact the first and second part. This advantageously provides a way of shielding the parts of the sensor that are not used in measuring load and ensures the measured load is applied to force-sensing parts of the sensor.

Any of the first part or the second part may comprise one or more slots for receiving the first sensor and the second sensor. The first part may comprise a cupped section and the second part may comprise a cupped section. The one or more slots may be disposed in the cupped section of the first part or the second part. The use of slots to secure the sensor array advantageously provides a passive way of securing the sensors to the device. Forming slots in the cupped sections also takes advantage of the existing material in the implant and therefore reduces the overall thickness of the implant as additional fixation materials or devices are not needed to secure the sensors array in place.

The cupped section comprising the one or more slots may have a first radius of curvature, and the one or more slots may have a second radius of curvature. The second radius of curvature may be greater than the first radius of curvature.

The device may comprise a processor operatively connected to the first sensor and the second sensor. The processor may be configured to: receive a first signal from the first sensor, receive a second signal from the second sensor, calculate a first value indicative of the first applied force, calculate a second value indicative of the second applied force, and output the first and second values.

The capacitive element of the first sensor may comprise a pair of electrodes. The capacitive element of the second sensor may comprise a pair of electrodes. The processor may be connected to the electrodes of the first and second sensors by a clamp.

Viewed from a further aspect, there is provided an orthopaedic implant comprising a device as described above. The orthopaedic implant may be any of an ankle, knee, hip, shoulder, elbow, spine, wrist or an interphalangeal implant. The device may be a human or animal orthopaedic implant.

Viewed from a further aspect, there is provided a method of manufacturing a micro- fluidic sensor, the method comprising: providing a first substrate, depositing a capacitive element onto the first substrate, providing a second substrate on the first substrate, wherein the first and second substrates define a cavity therebetween, the cavity defining a reservoir portion and a channel aligned with the capacitive element, and introducing a dielectric liquid into the reservoir portion.

This advantageously provides a low-cost method of producing microfluidic sensors that can be easily tuned to provide a desired response for a desired loading range.

The method may comprise the step of depositing an insulative coating on the capacitive element.

The capacitive element may be deposited with a printer tip having a first diameter, and the insulative coating may be deposited with a printer tip having a second diameter larger than the first diameter. By using a wider printer tip, the capacitive element is fully covered by insulative coating when following the same path used to deposit the capacitive element.

The capacitive element and the insulative coating may be deposited by aerosol jet printing.

The step of providing the first substrate may comprise forming a mould on a transfer sheet before depositing an elastomeric material onto the mould. The mould may have a profile corresponding to the cavity.

The second substrate may be bonded to the first substrate using a primer and a silicone glue. This advantageously provides an adhesive that can form a fluid-tight seal between materials having different stiffnesses.

There is also provided a method of implanting an orthopaedic implant into a patient in need thereof, comprising positioning the orthopaedic implant at a joint within the patient, and assessing the balance of the joint using the orthopaedic implant.

### BRIEF DESCRIPTION OF THE DRAWINGS

Embodiments of the invention are further described hereinafter with reference to the accompanying drawings, in which:

FIG. 1 illustrates a perspective view of an exemplary microfluidic sensor;

FIG. 2 illustrates an underside view of the microfluidic sensor of FIG. 1;

FIG. 3 illustrates a scanning electron microscopy image of moulding material used to form the reservoir portion of the microfluidic sensor of FIG. 1;

FIG. 4 illustrates a representation of an exemplary method of manufacturing a microfluidic sensor;

FIG. 5 illustrates a schematic representation of an exemplary method of manufacturing a microfluidic sensor;

FIG. 6 illustrates a graph of capacitance against force for different exemplary microfluidic sensors;

FIG. 7 illustrates a schematic of an exemplary orthopaedic hip implant having an array of microfluidic sensors;

FIG. 8 illustrates an exploded view of the implant of FIG. 7

FIG. 9 illustrates a schematic representation of the implant of FIG. 7 in use;

FIG. 10 illustrates exemplary loading scenarios of the implant of FIG. 7.

### DETAILED DESCRIPTION

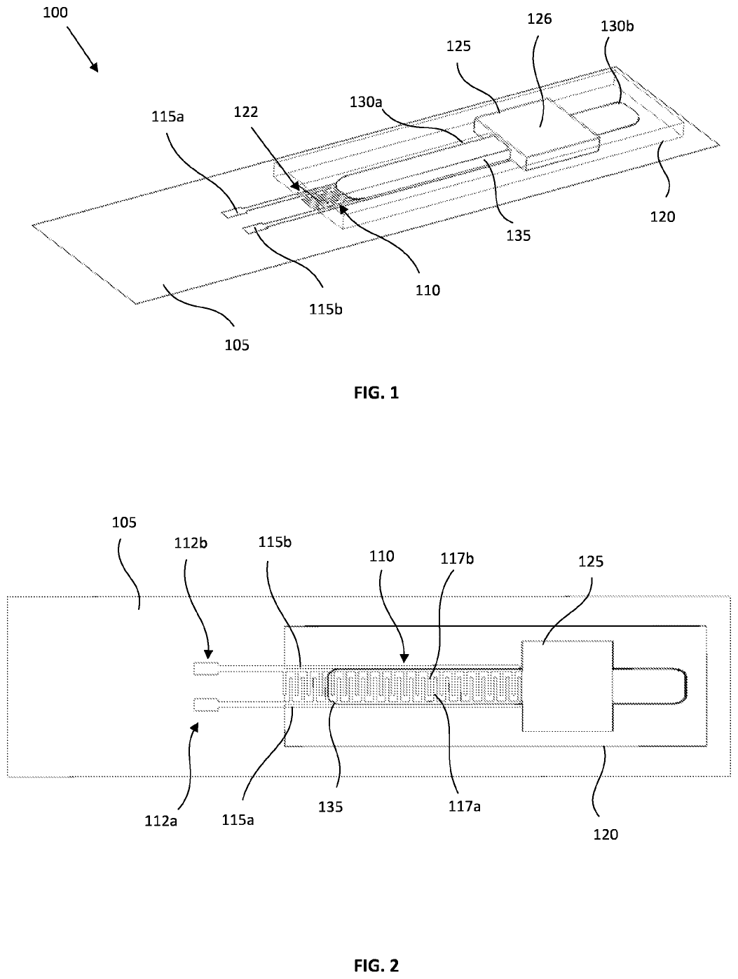

FIG. 1 illustrates a perspective view of an exemplary microfluidic sensor 100. The sensor 100 includes a polyimide layer 105 with a capacitive element 110 formed thereon and an elastomeric substrate 120 bonded to the polyimide layer 105. The elastomeric substrate 120 has a cavity formed therein for receiving a dielectric liquid 135. The cavity includes a first channel portion 130a, a second channel portion 130b and a reservoir portion 125 disposed between the first 130a and second 130b channel portions and allows fluid to pass from the second channel portion 130b to the first channel portion 130a. The capacitive element 110 is connected to first 115a and second 115b electrodes for connection with an external impedance analyser (not shown) for measuring the capacitance of the sensor 100.

The first channel portion 130a is preferably arranged over the capacitive element 110. The elastomeric substrate 120 also includes a force sensing surface 126 disposed on an external surface of the elastomeric substrate 120. The force sensing surface 126 is preferably arranged over the reservoir portion 125. The elastomeric substrate 120 also includes a fluid port 122 in fluid communication with the first channel portion 130a. The fluid port 122 allows fluid, such as air, to be expelled from within the cavity. A dielectric liquid 135 is disposed within the channel portions 130a, 130b and the reservoir portion 125. Upon application of a load to the force sensing surface 126, the elastomeric substrate 120 deforms, displacing the dielectric liquid 135 out of the reservoir portion 125 and along the first channel portion 130a in a longitudinal direction over the capacitive element 110. In an undeformed state of the microfluidic sensor 100, the dielectric liquid 135 is preferably contained within the reservoir portion 125. While it is preferable for no dielectric liquid 135 to be present within the channel portion 130a, it would be apparent that in some cases, a small amount of dielectric liquid 135 may be present within the channel portion 130a with at least some of the capacitive element 110 remaining uncovered by the dielectric liquid 135. In a deformed state of the microfluidic sensor 100, the dielectric liquid 135 is displaced along the channel portion 130a to change the capacitance of the capacitive element 110 within the channel portion 130a. Thus, while the force sensing surface 126 deforms relative to the polyimide layer 105, the constituent components of the capacitive element 110 remain in a fixed position relative to one another as load is applied to the force sensing surface 126 and as the dielectric liquid 135 is displaced along the channel portion 130a. This method of operation allows for a considerably larger range of forces to be measured compared to prior art sensors. Similarly, it is preferable that the elastomeric substrate 120 that makes up the channel portion 130a remains undeformed as the load sensing surface 126 is deformed. In the present microfluidic sensor 100, the dielectric environment above the capacitive element 110 is dependent on the coverage of the capacitive element 110. By deforming the load sensing surface 126, which is separate from the channel portion 130a, the volume of the reservoir portion 125 decreases and dielectric liquid 135 is displaced from the reservoir portion 125 and into the channel portion 130a. This increases the area of the capacitive element 110 covered by the dielectric liquid 135 which changes the dielectric environment of the capacitive element 110 in the channel portion 130a. That is to say, the present microfluidic sensor 100 measures a change in capacitance without changing the distance between the electrodes 115a, 115b. This advantageously provides a microfluidic sensor 100 which can sense a load independently of the distance between the electrodes 115a, 115b and is also independent of any reference pressure or capacitance values. As illustrated, the capacitive element 110 and the first channel portion 130a has substantially the same width perpendicular to the longitudinal direction. However, it would be apparent this was not essential, and that in some cases, the first channel portion 130a may have a width greater than that of the capacitive element 110. In some cases, the first channel portion 130a may have a width less than that of the capacitive element 110. In some cases the second channel portion 130b may be omitted entirely. The first 115a and second 115b electrodes and the capacitive element 110 preferably comprise silver.

The polyimide layer 105 is preferably formed as a film. While a polyimide layer 105 is described herein, it would be apparent this was merely an example of a suitable layer on which to deposit the capacitive element 110 and that other layers would be suitable. For example, the capacitive element 110 can be formed on a layer comprising any of a material having a Young's modulus between approximately 1 to 5 GPa, preferably 2 to 4 GPa, a thickness between approximately 50 μm to 100 μm, Kapton (polyimide), polyethylene terephthalate (PET), nylon and polymethylmethacrylate (PMMA) material. By providing a flexible substrate, the present microfluidic sensor 100 is highly flexible and can conform to a wide variety of shapes. Such a conformable microfluidic sensor 100 is particularly suited to orthopaedic applications where the geometry of a joint's surface may be highly irregular and/or non-planar. It is preferable that all parts of the capacitive element 110 are bonded to the polyimide layer 105.

The elastomeric substrate 120 preferably comprises polydimethylsiloxane (PDMS). However, additionally, or alternatively, the elastomeric substrate 120 may comprise any of polyurethane, a silicone material such as Ecoflex, low density polyethylene (LDPE), and any material having a Young's modulus between approximately 0.5 MPa to 500 MPa. The range of forces a given sensor 100 can measure has been found to be sensitive to the stiffness of the elastomeric substrate 120. Thus, by appropriately selecting the material for the elastomeric substrate 120, it is possible to “tune” the sensor 100 fora given force sensing application.

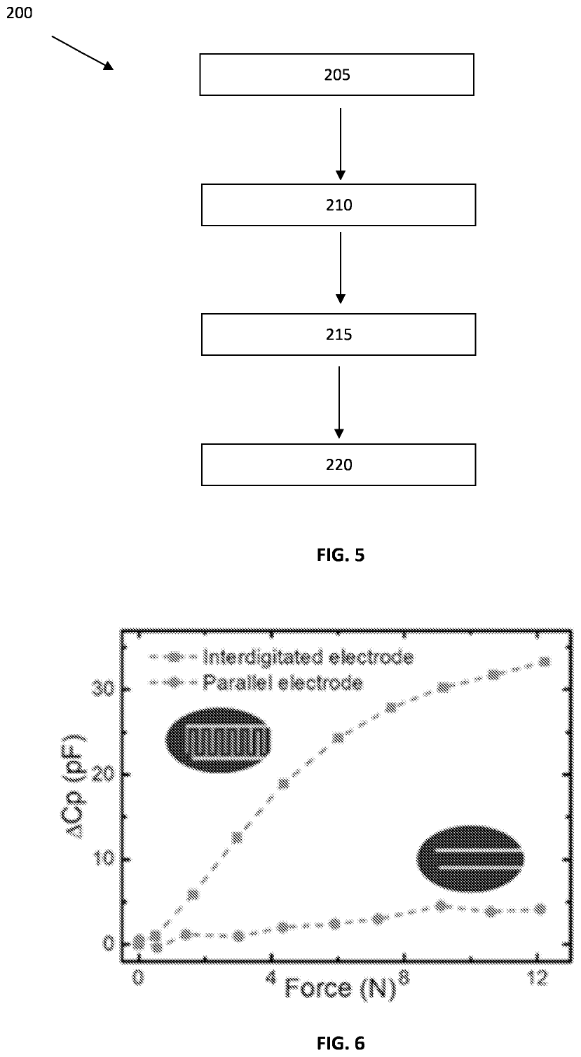

A dielectric liquid 135 comprising glycerol and deionised water at a 2:1 volume ratio has been found to be an effective working liquid for the present sensor. This ratio has been found to balance the volatility of the deionised water and the relatively low permittivity of pure glycerol (as compared with water). However, other dielectric liquids 135 would be suitable, such as phosphate-buffered saline (PBS). Preferably, the dielectric liquid 135 has a relative permittivity between approximately 10 and 100 so as to produce a target capacitance of less than 100 picofarads (see also FIG. 6). More preferably, the dielectric liquid 135 has a relative permittivity between approximately 30 and 80.

The illustrated reservoir portion 125 has a substantially square cross-sectional profile and the force sensing surface 126 has an area of approximately 4 mm². However, it would be apparent this was not essential, and the reservoir portion 125 may have other cross-sectional profiles, such as a substantially circular profile and be larger or smaller than 4 mm².

The present sensor is suited for many applications due to the range of materials it can be made from. For example, a polyimide layer 105 and an elastomeric substrate 120 comprising PDMS can be made into a sensor 100 having a width of 5 mm, a length of approximately 3 cm and a thickness of less than 1 mm. This allows the sensor to be easily bent into a concave or convex shape, which allows the present sensors to be used in a wide range of force-sensing applications, as each sensor can reliably measure up to 10 N of force. It would be apparent by changing one or more parameters of the geometry or the materials, this force sensing range could be manipulated as desired. By suitably adapting the geometry and material properties of the elastomeric substrate 120 it is possible to achieve a force sensing range in excess of 100 N.

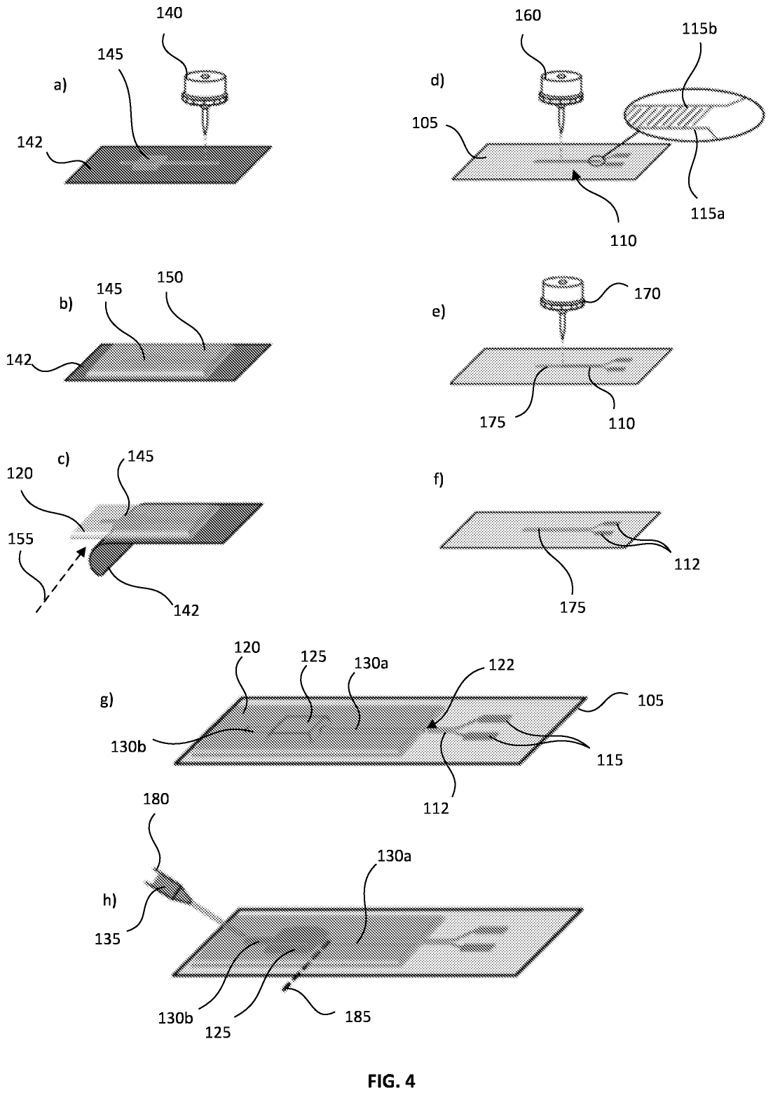

FIG. 2 illustrates an underside view of the microfluidic sensor 100 of FIG. 1. The illustrated capacitive element 110 includes a first electrode 115a and a second electrode 115b formed on the polyimide layer 105 that extend in a substantially linear direction under the first channel portion 130a. Each of the first 115a and second 115b electrodes also has a series of branches 117a, 117b extending therefrom in a perpendicular direction to the respective electrode 115a, 115b. The branches 117a, 117b are arranged to inter-digitate, such that branches 117a of the first electrode 115a inter-digitate with branches 117b of the second electrode 115b in an alternate manner. This greatly improves the sensitivity of the sensing element 110 compared to if there were no branches 117a, 117b. The sensing element 110 is also at least partially covered by an insulative coating 175 (see FIG. 4e). The insulative coating 175 provides a protective barrier by ensuring there is no physical contact between the dielectric liquid 135 and the covered portion of the sensing element 110. Thus, as the dielectric sensing liquid 135 is displaced along the first channel portion 130b, this causes a change in the permittivity within the first channel portion 130a which in turn results in a detectable change in capacitance of the material within the first channel portion 130a. The insulative coating 175 comprises any non-electrically conductive material that can be made into a liquid precursor that is stable against the particular dielectric liquid 135 used in the sensor 100. Preferably the liquid precursor has a viscosity less than 1000 centipoise (cP). Preferably the insulative coating 175 is a continuous film with a thickness of less than 20 μm. The insulative coating 175 may comprise any of polyimide (Kapton), polyvinylidene fluoride (PVDF), polyurethane, and nylon. By arranging the first 115a and second 115b electrodes on the polyimide layer 105 (i.e. the constituent parts of the capacitive element 110 are arranged on the polyimide layer 105), the distance between the first 115a and second 115b electrodes remains fixed throughout operation of the microfluidic sensor 100 as the force sensing surface 126 is deformed under an external load. It would also be apparent that it is not essential for the sensing element 110 to be restricted to the channel portion 130a, as illustrated in FIG. 2.

An end region 112a of the first electrode 115a and an end region 112b of the second electrode 115b are not covered by the insulative coating 175. This provides a convenient point from which to establish an electrical connection between the electrodes 115a, 115b and an impedance analyser used to measure the capacitance within the first channel portion 130a. By way of example, an impedance analyser (not shown) can be clamped to the electrodes 115a, 115b using a flexible printed circuit connector. To provide a secure connection with the flexible printed circuit, a portion of the polyimide layer 105 is cut out to correspond to the geometry of the flexible printed circuit connectors that are used to clamp the electrodes 115a, 115b. While an impedance analyser is described, it would be apparent that this was merely one apparatus suitable for connection with the sensor 100 and that other apparatuses would be equally suited.

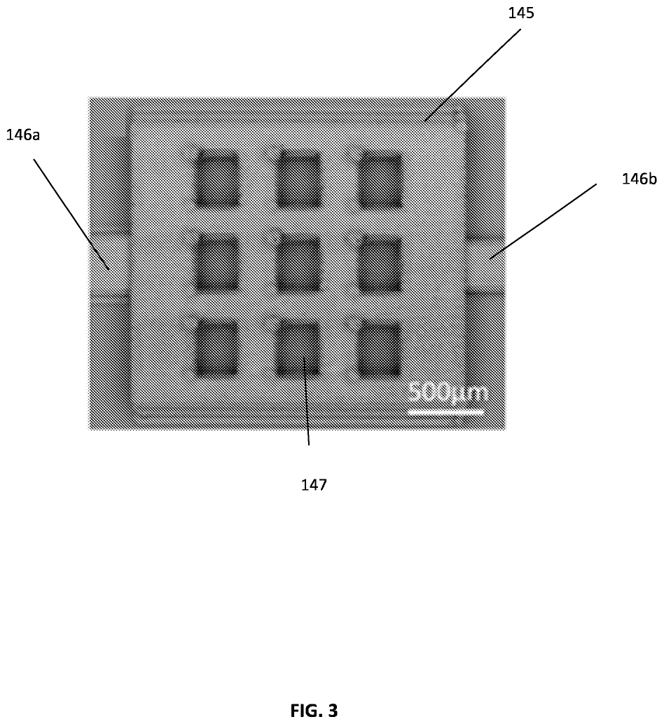

FIG. 3 illustrates a scanning electron microscopy image of a moulding material 145 used to form the reservoir portion 125 of the microfluidic sensor 100 (see also FIG. 4). The illustrated moulding material 145 has nine voids 147 and first 146a and second 146b branches. This produces a reservoir portion 125 with a corresponding profile having an array of nine wells and the first 130a and second 130b channel portions. The array of wells act to prevent the reservoir portion 125 collapsing under load. Preferably, the wells extend through the thickness of the elastomeric layer 120. In some cases, the wells extend from the elastomeric substrate 120 to the polyimide layer 105. While nine wells are illustrated it would be apparent more or fewer wells may be provided within the reservoir portion 125 by modifying the number or geometry of the voids 147 in the moulding material 145. For example, a single well may be used, or 16 wells may be included in the reservoir portion 125. The wells may be distributed so as to occupy between approximately 10% to 30% of the volume of the reservoir portion 125. It is also not essential for there to be any wells or supporting columns within the reservoir portion 125. In some cases, using a stiffer material than PDMS is sufficient to prevent collapse of the reservoir portion 125. This has the further advantage of improving the sensitivity of the sensor 100 compared to a reservoir portion 125 comprising wells or supporting columns.

FIG. 4 illustrates a representation of an exemplary method of manufacturing a microfluidic sensor. The illustrated method begins with depositing a moulding material 145 onto a transfer sheet 142 (FIG. 4a). The moulding material 145 is deposited in the shape of the cavity, preferably using an additive manufacture technique such as aerosol jet printing. Sodium chloride is one example of a moulding material that can be deposited as an “ink” onto the transfer sheet using a printer tip 140. The transfer sheet 142 may be an aluminium film. The use of additive manufacturing techniques allows for fine control over the geometry of the deposited moulding material 145, and thus the resulting cavity that is formed when the elastomeric substrate 120 is formed. In one example, the moulding material 145 is deposited as cuboid having dimensions of 2 mm×2 mm×0.3 mm (length x width x depth) and connected to an elongate section having dimensions of 20 mm×0.5 mm×0.2 mm (length x width x depth). The moulding material 145 may be deposited or shaped accordingly to provide the outline of any wells that are to be present in the elastomeric substrate 120. It would be apparent that aerosol jet printing was merely provided as an exemplary method of depositing the moulding material 145 and that other techniques would be suitable. For example depositing the moulding material 145 using a 3D printer would be suitable. If a 3D printed mould is used, it is not necessary to provide a transfer sheet 142.

Liquid material 150 can then be poured on top of the moulding material 145 and cured (FIG. 4b) to form the elastomeric substrate 120. The transfer sheet 142 can then be peeled away from the elastomeric substrate 120 to expose the moulding material 145 (FIG. 4c) within the cavity so that the moulding material 145 can then be washed away using a fluid stream 155 to provide the resulting elastomeric substrate 120 having the desired cavity The distal end of the first channel portion 130a (i.e. the end opposite to the reservoir portion 125) has a fluid port 122 that is open to the air. This allows the pressure of the dielectric liquid 135 to remain relatively constant as the load sensing surface 126 is deformed and as dielectric liquid 135 is displaced through the channel portion 130a. It would also be apparent that it is possible to 3D print the elastomeric substrate 120 in the desired geometry, thus avoiding the steps of depositing the moulding material 145 and pouring the liquid material 150 onto the moulding material 145 to form the elastomeric substrate 120.

By way of example, the electrodes 115a, 115b and branches 117a, 117b are formed on the polyimide layer 105 by depositing silver using aerosol jet printing. In one case, the electrodes 115a, 115b and branches 117a, 117b are deposited using a printer tip 160 (FIG. 4d) to form the sensing element 110. A second printer tip 170 is then used to deposit the insulative coating 175 on top of the sensing element 110. Preferably, the insulative coating 175 is deposited in the same pattern as the sensing element 110 (FIG. 4e). By using a wider printer tip (for example, a 300 μm tip compared to a 150 μm tip used for depositing the electrodes 115a, 115b), the liquid insulative material can flow over and around the area of the electrodes 115a, 115b and branches 117a, 117b, thus ensuring the electrodes 115a, 115b and branches 117a, 117b are fully covered by the insulative coating 175, with the exception of the ends 112a, 112b which are left uncovered for establishing an electrical connection as described above (FIG. 4f). The printer tip 160 for printing the electrodes 115 may have a diameter between 100 μm and 150 μm. The printer tip 170 for printing the insulative coating 175 may have a diameter between 250 μm to 300 μm.

The elastomeric substrate 120 is then attached to the polyimide layer 105 using a glue to establish a fluid-tight seal (FIG. 4g). The glue may comprise a primer and a silicone glue. While the combination of a primer and a silicone glue is described, it would be apparent that both are not essential. In some cases, a silicone glue may be used to bond the polyimide layer 105 to the elastomeric substrate 120 without the primer . However, when the elastomeric substrate 120 is to be bonded to a layer that is stiffer than the elastomeric substrate 120, a primer can be used to modify the surface property of the stiffer layer to provide good adhesion with silicone glue. As such, a primer advantageously allows for a softer material, such as silicone, to be adhered to stiffer materials, such as the polyimide layer 105.

When attaching the polyimide layer 105 to the elastomeric substrate 120, the first channel portion 130a is aligned with the interdigitated branches 117a, 117b (FIG. 2). This may be done under a microscope to facilitate proper alignment. The sensor 100 can then be optionally tailored into a suitable profile using manual or automated cutting techniques, such as using a scalpel or a laser cutter. Once the desired shape of the sensor 100 has been achieved, the dielectric liquid 135 can be introduced into the reservoir portion 125. As illustrated in FIG. 4h, one way of achieving this is to inject the dielectric liquid 135 into the reservoir portion 125 through the second channel portion 130b until the dielectric liquid 135 fills the second channel portion 130b and the reservoir portion 125. As shown in FIG. 4h, filling the first channel portion 130a up to a fill line 185 is one way to ensure the reservoir portion 125 is completely filled. The injection hole is then sealed with a tape, such as a polyimide tape. Alternatively, the injection hole can be sealed with a silicone glue. The electrodes 115a, 115b can then be connected for capacitance measurements using a flexible printed circuit connector (not shown).

The method of manufacturing described above and illustrated in FIG. 4, can be generalised as shown in FIG. 5 to include the steps of providing 205 a polyimide layer 105, depositing 210 a capacitive element 110 onto the polyimide layer 105, providing 215 the elastomeric substrate 120 on the polyimide layer 105, introducing 220 the dielectric liquid 135 into the reservoir portion 125.

A typical force-capacitance measurement obtained from a sensor of the present application is shown in FIG. 6. As the load applied to the reservoir portion 125 is increased, there is a linear relationship between the applied force and the corresponding change in capacitance up to approximately 9 N. This linear relationship demonstrates a sensor with a sensitivity of 3.7 pF/N. This linear relationship reflects the elastic deformation of the elastomeric substrate 120, which decreases the volume within the reservoir portion 125 and leads to a corresponding change of the capacitance between the electrodes 115a, 115b as the dielectric liquid 135 is displaced through the first channel portion 130a. By way of comparison, parallel electrodes (lower line in FIG. 6) show a considerably reduced sensitivity of 0.55 pF/N.

Furthermore, by modifying the geometry of the reservoir portion 125 and/or the first channel portion 130a, it is possible to change the sensitivity and/or the measurable force range of a given sensor. For example, for a given size of reservoir portion 125, decreasing the width of the first channel portion 130a such that it is narrower than the width of the inter-digitated portion of the capacitive element 110 shows similar sensitivity to where the first channel portion 130a and the inter-digitated portion of the capacitive element 110 have the same width. However, the force detection range is smaller, as less deformation of the reservoir portion 125 is required to displace the dielectric liquid 135 to the fluid port 122. Conversely, when the first channel portion 130a is wider than the inter-digitated portion of the capacitive element 110, a larger measurement range is achieved but with less sensitivity, since the volume of dielectric liquid 135 covering a unit portion of the capacitive element 110 is larger compared to when the first channel portion 130a is narrower. Similarly, by modifying the thickness of the elastomeric substrate 120 above the reservoir portion 125, it is possible to alter the sensitivity of the sensor 100. For example, a thicker elastomeric substrate 120 has increased stiffness, and will therefore deform less under a given load. This will lead to smaller volume decreases within the reservoir portion 125 for a given load, which results in an increased measurement range, with reduced sensitivity of the sensor 100. This effect has been found for an elastomeric substrate 120 having a thickness between 0.5 mm and 2 mm. The force sensing surface 126 may have an area between 10 and 100 times the cross-sectional area of the first channel portion 130a that is perpendicular to the direction of fluid flow.

A given sensor 100 will have specific geometric and material properties, and will therefore require calibration in order to determine the force-capacitance relationship of the particular sensor. The calibration process involves applying a known force to the sensor 100 and measuring the resulting capacitance between the electrodes 115a, 115b. A calibration curve can therefore be established for each sensor in order to be able to subsequently measure an unknown load that is applied to the sensor 100 during use. Preferably, the calibration data used to determine the applied forces are the linear region of the force-capacitance curve shown in FIG. 6. The capacitance measured by the present sensor is governed by the changing volume of the dielectric liquid within the first channel portion 130a. This approach advantageously does not require direct contact between the dielectric liquid and the electrodes 115a, 115b. An impedance analyser can be used to measure the capacitance response of the sensor as an unknown force is applied to the sensor 100. The capacitance value is then converted into a measurement of force using the previously-obtained calibration data.

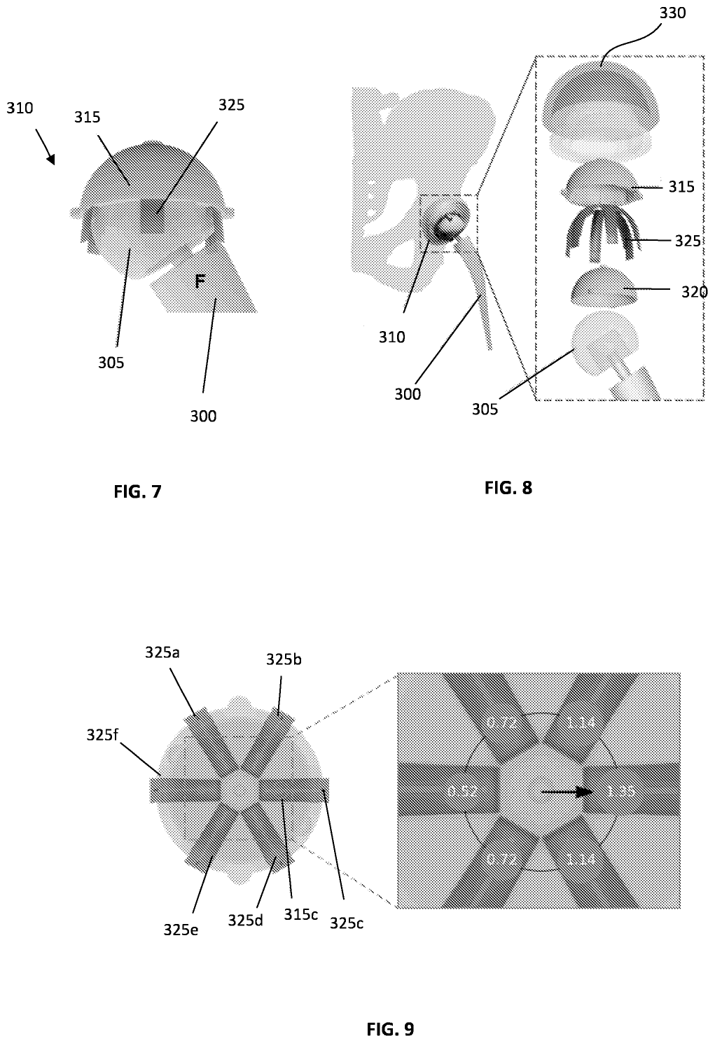

FIG. 7 illustrates a schematic of an exemplary orthopaedic hip implant 310 having an array of microfluidic sensors 325. Reference will also be made to FIG. 8. The instrumented hip implant 310 includes an inner cup 320, an array of microfluidic sensors 325 arranged in a radial manner and an outer cup 315. The outer cup 315 is secured to an acetabular cup 330 that is to be implanted into a patient. The outer cup 315 is also shaped to receive the inner cup 320 and the sensor array 325, and includes one or more slots for receiving one or more of the sensors of the sensor array 325. The slots are arranged on an inner surface of the outer cup 315 such that the reservoir portion 125 of each sensor of the sensor array 325 protrudes beyond the inner surface of the outer cup 315. This ensures the remaining parts of the sensor 100, such as the respective first channel portion 130a, are shielded from the loads that are applied between the inner 320 and outer 315 cups. By having only the reservoir portions 125 disposed in the gap between the inner 320 and outer 315 cups, this ensures the load applied by the femoral head 305 is transmitted to the respective force sensing surfaces 126 of each sensor 100, and not to other parts of the sensor. One way to achieve this is to provide an outer cup 315 with an inner surface having a first radius of curvature and have slots with a second radius of curvature that is larger than the first radius of curvature. This creates a slot with a reducing depth as you travel from the periphery of the outer cup 315 towards the centre. Each sensor of the sensor array 325 is arranged such that the reservoir portions (e.g. 315c of sensor 325c) are all located adjacent the centre of the outer cup 315 (i.e. the flow direction within each channel portion is away from the centre of the outer cup 315). When assembled, the tapering slots will raise the reservoir portions 125 out of the slot and into the gap. While the slots have been described as being formed in the outer cup 315, it would be apparent this was not essential, and that in some cases, some or all of the slots may be formed in an outer surface of the inner cup 320 in order to provide the necessary recesses for receiving and shielding the sensor array 325.

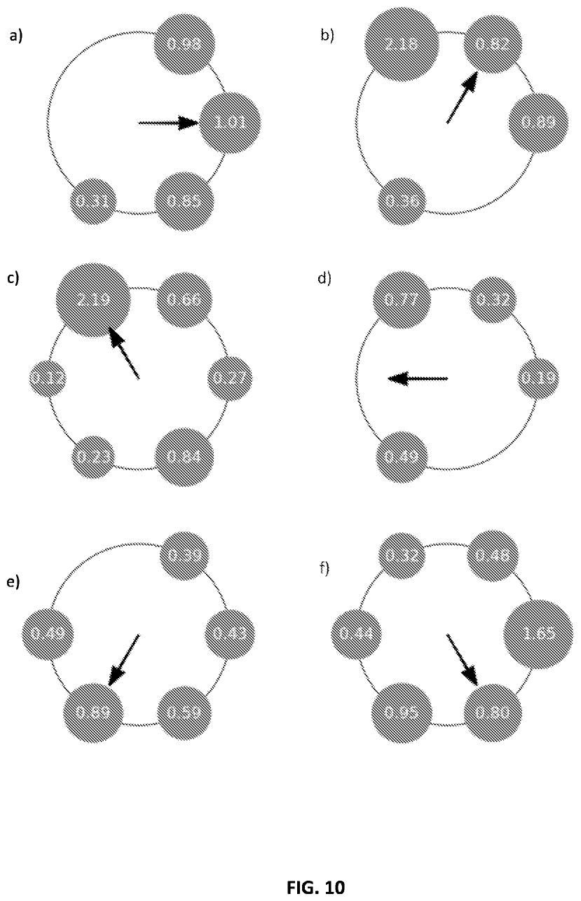

As shown in FIGS. 9 and 10, the array of sensors 325 provides a way of determining a net force that is applied between a femoral head 305 and an acetabular cup 330. By mapping the forces measured at each of the different locations of the respective reservoir portions 125 in the hip implant 310, it is possible to map the distribution of forces within the implant 310 and derive the direction and magnitude of the net force. The numerical value shown in the Figures correspond to the ratio of measured load to the known load that was applied to the femoral head. The different loading scenarios illustrated in FIG. 10 correspond to applying the known load at 10 degrees relative to the vertical axis, with the implant 310 secured in a horizontal orientation with the periphery of the outer cup 315 forming a substantially horizontal plane. While six sensors are illustrated, it would be apparent more or fewer than six sensors may be used. The number and arrangement of sensors will also depend on the specific application.

Such an implant would be particularly advantageous in an orthopaedic setting, as an instrumented component can help a surgeon objectively measure how balanced a particular joint is, and whether the position of any of the components needs to be modified. By determining the net force and the magnitude of the force at different orientations, in a similar manner to that illustrated in FIG. 10, the present sensor can help to inform whether or not the joint is balanced. Where an instrumented hip implant is implanted into the patient, this would provide longer term monitoring of the in vivo joint loading. To facilitate data communication from the implant 310, the implant 310 may include a communication module for sending loading data to an external receiver. The external receiver may be connected to a display for displaying the received data.

While a hip implant has been described, it would be apparent the present implant arrangement is suitable for use in other ball and socket joints (such as the shoulder), or hinge joints (such as the elbow, knee or ankle) or the small joints of the hands or feet (such as the interphalangeal joints of the hand and feet). Whilst it may not be essential to incorporate a hemispherical shell, as shown in FIG. 7, the implant may still incorporate two cupped shaped portions. This would allow for the sensor array to be secured within the device in the manner described above. The cupped shaped portions may subtend an angle between 10 and 80 degrees in order to provide a pair of surfaces that can cooperate in the desired manner in a range of loading applications. Similarly, while the sensor has been described in the context of instrumented orthopaedic implants, it would be apparent this was merely one type of device that benefits from the present microfluidic sensor 100. Other devices outside of orthopaedic implants, such as footwear or sports equipment, that have previously been instrumented for force measurement would provide other applications that would benefit from the versatility of the described microfluidic sensor 100.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A microfluidic sensor comprising:
a first substrate;
a second substrate;
a cavity formed between the first substrate and the second substrate, the cavity comprising a reservoir portion and a channel portion extending from the reservoir portion;
a capacitive element disposed between the first substrate and the second substrate, the capacitive element being at least partially disposed in the channel portion of the cavity; and
a dielectric sensing liquid provided in the reservoir portion;
wherein, upon application of a force to the second substrate adjacent the reservoir portion, the reservoir portion is configured to deform and displace the sensing liquid along the channel portion, so as to change the capacitance of the capacitive element.

2. A sensor according to claim 1, wherein the sensing liquid comprises a liquid having a relative permittivity of between 10 and 100.

3. A sensor according to claim 1 comprising an insulative coating disposed on a portion of the capacitive element.

4. A sensor according to claim 1, wherein the reservoir portion has a cross-sectional area between approximately 10 and 100 times greater than a cross-sectional area of the channel portion.

5. A sensor according to claim 1, wherein the capacitive element is formed on a single surface of the channel portion.

6. A sensor according to claim 5, wherein the capacitive element comprises:
a first electrode extending from a first end to a second end and having a plurality of branches extending therefrom between the first end and the second end, and
a second electrode extending from a first end to a second end and having a plurality of branches extending therefrom between the first end and the second end, and
wherein the plurality of branches of the first electrode are arranged to inter-digitate with the plurality of branches of the second electrode within the channel portion.

7. A sensor according to claim 1, comprising at least one resiliently deformable member extending between the first and second substrates in the reservoir portion.

8. (canceled)

9. A sensor according to claim 1, wherein the channel portion extends from the reservoir portion to a distal end, and wherein the sensor comprises a fluid port at the distal end.

10. A device comprising:
a first sensor according to claim 1 and configured to detect a first force applied at a first position on the device, and
a second sensor according to claim 1 and configured to detect a second force applied at a second position on the device.

11. (canceled)

12. A device according to claim 10 comprising:
a first part and
a second part configured to receive at least a portion of the first part, such that when the portion of the first part is received within the second part, a gap is defined between the first part and the second part,
wherein, in use, the reservoir portion of the first sensor and the reservoir portion of the second sensor are disposed in the gap and are arranged to contact the first and second part.

13. A device according to claim 12, wherein any of the first part or the second part comprises one or more slots for receiving the first sensor and the second sensor.

14. A device according to claim 13, wherein the first part comprises a cupped section and the second part comprises a cupped section, and wherein the one or more slots are disposed in the cupped section of the first part or the second part.

15. (canceled)

16. A device according to claim 10 comprising a processor operatively connected to the first sensor and the second sensor, wherein the processor is configured to
receive a first signal from the first sensor,
receive a second signal from the second sensor,
calculate a first value indicative of the first applied force,
calculate a second value indicative of the second applied force, and
output the first and second values.

17. A device according to claim 16, wherein the capacitive element of the first sensor comprises a pair of electrodes, wherein the capacitive element of the second sensor comprises a pair of electrodes, and wherein the processor is connected to the electrodes of the first and second sensors by a clamp.

18. An orthopaedic implant comprising a device according to claim 10.

19. (canceled)

20. A method of manufacturing a micro-fluidic sensor, the method comprising:
providing a first substrate,
depositing a capacitive element onto the first substrate,
providing a second substrate on the first substrate, wherein the first and second substrates define a cavity therebetween, the cavity defining a reservoir portion and a channel aligned with the capacitive element, and
introducing a dielectric liquid into the reservoir portion.

21. A method according to claim 20 comprising depositing an insulative coating on the capacitive element.

22. A method according to claim 21, wherein the capacitive element is deposited with a printer tip having a first diameter, and wherein the insulative coating is deposited with a printer tip having a second diameter larger than the first diameter.

23. (canceled)

24. A method according to claim 20, wherein the step of providing the first substrate comprises forming a mould on a transfer sheet before depositing an elastomeric material onto the mould, and wherein the mould has a profile corresponding to the cavity.

25. (canceled)

26. A method of implanting an orthopaedic implant according to claim 18 into a patient in need thereof, comprising: positioning the orthopaedic implant at a joint within the patient, and assessing the balance of the joint using the orthopaedic implant.
